(19) United States
(12) Patent Application Publication
(10) Pub. No.: US 2026/0120795 A1
(43) Pub. Date: Apr. 30, 2026

(54) TECHNIQUES FOR COMPUTATIONAL TARGET IDENTIFICATION AND VALIDATION

(71) Applicant: DEEP FOREST SCIENCES, INC., Palo Alto, CA (US)

(72) Inventors: ARYAN AMIT BARSAINYAN, Bellary (IN); BHARATH RAMSUNDAR, Palo Alto, CA (US)

(73) Assignee: DEEP FOREST SCIENCES, INC., Palo Alto, CA (US)

(21) Appl. No.: 19/375,155

(22) Filed: Oct. 30, 2025

Related U.S. Application Data
(60) Provisional application No. 63/714,120, filed on Oct. 30, 2024.

(51) Int. Cl. G16B 15/30; G16B 15/20; G16B 40/20

(52) U.S. Cl. CPC G16B 15/30; G16B 15/20; G16B 40/20

(57) ABSTRACT

Various aspects of the present disclosure relate to techniques for computational target identification and validation. An apparatus is configured to determine protein structure data associated with a plurality of genes, wherein the protein structure data describes one or more protein structures, simulate interactions between a plurality of compounds and the one or more protein structures, determine binding affinities between each of the plurality of compounds and the one or more protein structures, and generate a list of validated or novel targets based on the binding affinities.

### CROSS REFERENCE TO RELATED APPLICATIONS

This application claims the benefit of U.S. Provisional Patent Application No. 63/714,120 entitled “TECHNIQUES FOR TARGET IDENTIFICATION AND VALIDATION ENGINE” and filed on Oct. 30, 2024, for Aryan Amit Barsainyan, et al., which is incorporated herein by reference.

### FIELD

The subject matter herein relates generally to computational biology and bioinformatics, and more particularly to computational biology and bioinformatics, and more particularly to techniques for identifying and validating biological targets using computer-implemented molecular modeling and analysis.

### BACKGROUND

Drug discovery and development traditionally require identification of biological targets, such as proteins or genes, that are associated with a particular disease or physiological process. Confirming that modulation of a given target produces a desired therapeutic effect—commonly referred to as target validation—typically involves extensive experimental testing and clinical evaluation, which are both costly and time-consuming. As a result, there is a growing need for computational systems and methods that can efficiently analyze biological data, simulate compound-target interactions, and predict promising therapeutic or toxicological targets before experimental validation.

### SUMMARY

In one embodiment, an apparatus is configured to determine protein structure data associated with a plurality of genes, wherein the protein structure data describes one or more protein structures; simulate interactions between a plurality of compounds and the one or more protein structures; determine binding affinities between each of the plurality of compounds and the one or more protein structures; and generate a list of validated or novel targets based on the binding affinities.

In one embodiment, a method includes determining protein structure data associated with a plurality of genes, wherein the protein structure data describes one or more protein structures, simulating interactions between a plurality of compounds and the one or more protein structures, determining binding affinities between each of the plurality of compounds and the one or more protein structures, and generating a list of validated or novel targets based on the binding affinities.

In one embodiment, a computer program product comprises a non-transitory computer-readable medium storing instructions that, when executed by one or more processors, cause the one or more processors to determine protein structure data associated with a plurality of genes, wherein the protein structure data describes one or more protein structures, simulate interactions between a plurality of compounds and the one or more protein structures, determine binding affinities between each of the plurality of compounds and the one or more protein structures, and generate a list of validated or novel targets based on the binding affinities.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a schematic block diagram illustrating one embodiment of a system in accordance with the subject matter disclosed herein;

FIG. 2 illustrates one example of an apparatus in accordance with the subject matter disclosed herein;

FIG. 3 illustrates an example embodiment of a gene-based docking workflow in accordance with the subject matter disclosed herein;

FIG. 4 illustrates an example embodiment of a large-scale docking workflow in accordance with the subject matter disclosed herein;

FIG. 5 illustrates a flowchart showing one example of a method in accordance with the subject matter disclosed herein; and

FIG. 6 illustrates a flowchart showing one example of a method in accordance with the subject matter disclosed herein.

### DETAILED DESCRIPTION

Many therapeutic agents act by binding to and modulating specific biological targets such as proteins or enzymes. The identification and validation of these targets—often referred to as “target validation”—are critical steps in drug discovery and development. Conventionally, confirming a target's role in a disease involves extensive laboratory experimentation and clinical validation, processes that are costly, time-consuming, and limited in scalability. As a result, many promising targets remain unverified or are discarded prematurely, slowing the pace of therapeutic innovation.

Computational biology has introduced techniques such as molecular docking and in silico screening to accelerate this process. However, traditional computational approaches often rely on incomplete or isolated protein models, failing to capture the full diversity of protein conformations or gene variants relevant to biological function. Furthermore, most existing docking pipelines are optimized for evaluating a small number of compounds against a single protein, rather than systematically analyzing large-scale gene-based interactions. This fragmentation makes it difficult to determine whether a compound's observed biological effects arise from intended or off-target interactions.

The techniques described in this disclosure address these limitations through a computational target identification and validation engine configured to perform large-scale, gene-based docking and interaction analysis. The disclosed solution retrieves, prepares, and analyzes protein structure data from curated biological databases, selects representative structures for each gene based on sequence coverage and resolution, and performs high-exhaustiveness docking simulations between multiple compounds and corresponding protein structures. The results are analyzed to determine binding affinities and to generate ranked lists of validated or novel biological targets.

In one embodiment, the system executes the docking computations in parallel across multiple processors or servers, enabling high-throughput analysis of thousands of gene-compound pairs. In another embodiment, the system incorporates machine learning models trained to identify statistically significant or biologically meaningful interaction patterns, including promiscuous targets that bind multiple compounds or previously unrecognized off-target effects. The resulting framework enables continuous refinement of target predictions and supports integration with downstream experimental or clinical validation workflows.

Accordingly, the disclosed techniques provide a scalable, automated, and reproducible solution for computational target validation. By integrating data acquisition, structural optimization, parallel docking, and AI-driven filtering, the system reduces manual intervention, increases confidence in predicted targets, and accelerates the discovery of therapeutic mechanisms across a wide range of disease domains.

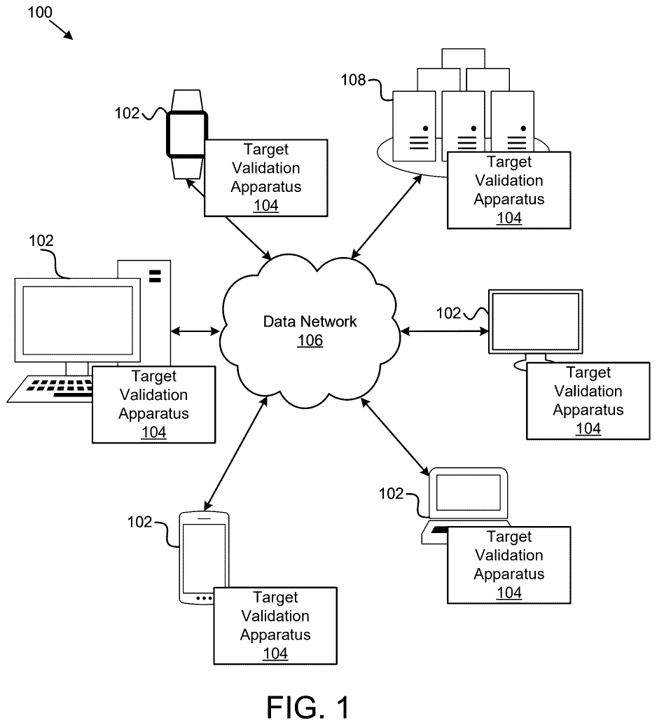

FIG. 1 is a schematic block diagram illustrating one embodiment of a system 100 for techniques for computational target identification and validation. In one embodiment, the system 100 includes one or more information handling devices 102, one or more target validation apparatuses 104, one or more data networks 106, and one or more servers 108. In certain embodiments, even though a specific number of information handling devices 102, target validation apparatuses 104, data networks 106, and servers 108 are depicted in FIG. 1, one of skill in the art will recognize, in light of this disclosure, that any number of information handling devices 102, target validation apparatuses 104, data networks 106, and servers 108 may be included in the system 100.

In one embodiment, the system 100 includes one or more information handling devices 102. The information handling devices 102 may be embodied as one or more of a desktop computer, a laptop computer, a tablet computer, a smart phone, a smart speaker (e.g., Amazon Echo®, Google Home®, Apple HomePod®), an Internet of Things device, a security system, a set-top box, a gaming console, a smart TV, a smart watch, a fitness band or other wearable activity tracking device, an optical head-mounted display (e.g., a virtual reality headset, smart glasses, head phones, or the like), a High-Definition Multimedia Interface (“HDMI”) or other electronic display dongle, a personal digital assistant, a digital camera, a video camera, or another computing device comprising a processor (e.g., a central processing unit (“CPU”), a processor core, a field programmable gate array (“FPGA”) or other programmable logic, an application specific integrated circuit (“ASIC”), a controller, a microcontroller, and/or another semiconductor integrated circuit device), a volatile memory, and/or a non-volatile storage medium, a display, a connection to a display, and/or the like.

In one embodiment, the target validation apparatus 104 is configured to execute the core computational functions of the target identification and validation process. The target validation apparatus 104 includes at least one memory and at least one processor coupled thereto and is configured to determine or retrieve protein structure data associated with a plurality of genes, wherein the protein structure data describes one or more protein structures obtained from biological databases. The target validation apparatus 104 is further configured to simulate interactions between a plurality of compounds and the one or more protein structures using a molecular docking or equivalent computational chemistry process. Based on the results of these simulations, the target validation apparatus 104 is configured to determine binding affinities between each of the plurality of compounds and the one or more protein structures, such as by evaluating computed docking scores or binding energies. The target validation apparatus 104 is additionally configured to generate a list of validated or novel targets based on the binding affinities, such that the resulting data identify one or more genes or proteins likely to represent valid therapeutic or toxicological targets. In some embodiments, the target validation apparatus 104 may further perform auxiliary operations including selection of optimal structure files based on coverage and resolution, execution of docking computations in parallel across multiple processing units or servers, identification and exclusion of promiscuous targets, and application of trained machine-learning models to predict off-target interactions or toxicity indicators.

In certain embodiments, the target validation apparatus 104 may include a hardware device such as a secure hardware dongle or other hardware appliance device (e.g., a set-top box, a network appliance, or the like) that attaches to a device such as a head mounted display, a laptop computer, a server 108, a tablet computer, a smart phone, a security system, a network router or switch, or the like, either by a wired connection (e.g., a universal serial bus (“USB”) connection) or a wireless connection (e.g., Bluetooth®, Wi-Fi, near-field communication (“NFC”), or the like); that attaches to an electronic display device (e.g., a television or monitor using an HDMI port, a DisplayPort port, a Mini DisplayPort port, VGA port, DVI port, or the like); and/or the like. A hardware appliance of the target validation apparatus 104 may include a power interface, a wired and/or wireless network interface, a graphical interface that attaches to a display, and/or a semiconductor integrated circuit device as described below, configured to perform the functions described herein with regard to the target validation apparatus 104.

The target validation apparatus 104, in such an embodiment, may include a semiconductor integrated circuit device (e.g., one or more chips, die, or other discrete logic hardware), or the like, such as a field-programmable gate array (“FPGA”) or other programmable logic, firmware for an FPGA or other programmable logic, microcode for execution on a microcontroller, an application-specific integrated circuit (“ASIC”), a processor, a processor core, or the like. In one embodiment, the target validation apparatus 104 may be mounted on a printed circuit board with one or more electrical lines or connections (e.g., to volatile memory, a non-volatile storage medium, a network interface, a peripheral device, a graphical/display interface, or the like). The hardware appliance may include one or more pins, pads, or other electrical connections configured to send and receive data (e.g., in communication with one or more electrical lines of a printed circuit board or the like), and one or more hardware circuits and/or other electrical circuits configured to perform various functions of the target validation apparatus 104.

The semiconductor integrated circuit device or other hardware appliance of the target validation apparatus 104, in certain embodiments, includes and/or is communicatively coupled to one or more volatile memory media, which may include but is not limited to random access memory (“RAM”), dynamic RAM (“DRAM”), cache, or the like. In one embodiment, the semiconductor integrated circuit device or other hardware appliance of the target validation apparatus 104 includes and/or is communicatively coupled to one or more non-volatile memory media, which may include but is not limited to: NAND flash memory, NOR flash memory, nano random access memory (nano RAM or “NRAM”), nanocrystal wire-based memory, silicon-oxide based sub-10 nanometer process memory, graphene memory, Silicon-Oxide-Nitride-Oxide-Silicon (“SONOS”), resistive RAM (“RRAM”), programmable metallization cell (“PMC”), conductive-bridging RAM (“CBRAM”), magneto-resistive RAM (“MRAM”), dynamic RAM (“DRAM”), phase change RAM (“PRAM” or “PCM”), magnetic storage media (e.g., hard disk, tape), optical storage media, or the like.

The data network 106, in one embodiment, includes a digital communication network that transmits digital communications. The data network 106 may include a wireless network, such as a wireless cellular network, a local wireless network, such as a Wi-Fi network, a Bluetooth® network, a near-field communication (“NFC”) network, an ad hoc network, and/or the like. The data network 106 may include a wide area network (“WAN”), a storage area network (“SAN”), a local area network (“LAN”) (e.g., a home network), an optical fiber network, the internet, or other digital communication network. The data network 106 may include two or more networks. The data network 106 may include one or more servers, routers, switches, and/or other networking equipment. The data network 106 may also include one or more computer readable storage media, such as a hard disk drive, an optical drive, non-volatile memory, RAM, or the like.

The wireless connection may be a mobile telephone network. The wireless connection may also employ a Wi-Fi network based on any one of the Institute of Electrical and Electronics Engineers (“IEEE”) 802.11 standards. Alternatively, the wireless connection may be a Bluetooth® connection. In addition, the wireless connection may employ a Radio Frequency Identification (“RFID”) communication including RFID standards established by the International Organization for Standardization (“ISO”), the International Electrotechnical Commission (“IEC”), the American Society for Testing and Materials® (ASTM®), the DASH7™ Alliance, and EPCGlobal™.

Alternatively, the wireless connection may employ a ZigBee® connection based on the IEEE 802 standard. In one embodiment, the wireless connection employs a Z-Wave® connection as designed by Sigma Designs®. Alternatively, the wireless connection may employ an ANT® and/or ANT+® connection as defined by Dynastream® Innovations Inc. of Cochrane, Canada.

The wireless connection may be an infrared connection including connections conforming at least to the Infrared Physical Layer Specification (“IrPHY”) as defined by the Infrared Data Association® (“IrDA” ). Alternatively, the wireless connection may be a cellular telephone network communication. All standards and/or connection types include the latest version and revision of the standard and/or connection type as of the filing date of this application.

The one or more servers 108, in one embodiment, may be embodied as blade servers, mainframe servers, tower servers, rack servers, and/or the like. The one or more servers 108 may be configured as mail servers, web servers, application servers, FTP servers, media servers, data servers, web servers, file servers, virtual servers, and/or the like. The one or more servers 108 may be communicatively coupled (e.g., networked) over a data network 106 to one or more information handling devices 102 and may be configured to execute or run machine learning algorithms, programs, applications, processes, and/or the like.

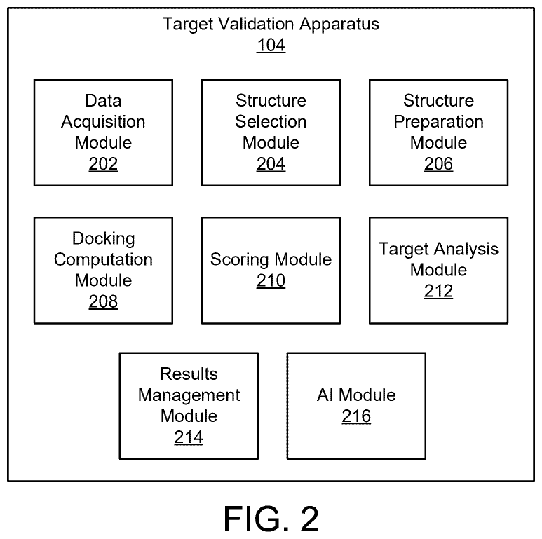

FIG. 2 depicts one embodiment of an apparatus for computational target identification and validation. In one embodiment, the apparatus includes an instance of a target validation apparatus 104. The target validation apparatus 104, in one embodiment, includes one or more of a data acquisition module 202, a structure selection module 204, a structure preparation module 206, a docking computation module 208, a scoring module 210, a target analysis module 212, a results management module 214, and an AI module 216. Each module may be implemented as software instructions executed by one or more processors, hardware logic, firmware, or any combination thereof.

In one embodiment, the data acquisition module 202 is configured to obtain, aggregate, and manage biological and chemical data necessary for the computational target identification and validation process. The data acquisition module 202 retrieves protein sequence and structure data associated with a plurality of genes from one or more curated biological databases, such as UniProt, PDBe-KB, Protein Data Bank (PDB), Ensembl, or other publicly or privately maintained genomic and proteomic data repositories. Each protein entry retrieved by the data acquisition module 202 may include a unique amino acid sequence, gene association information, and one or more experimental or computationally derived structural conformations.

The data acquisition module 202, in one embodiment, accesses these data sources via a network interface coupled to the target validation apparatus 104 or a remote server 108 over the data network 106. The data acquisition module 202 may employ application programming interfaces (APIs), structured query language (SQL) queries, or web-based retrieval mechanisms to collect protein data in standard formats such as FASTA, PDB, or mmCIF. Once retrieved, the data acquisition module 202 normalizes the data into a consistent internal schema, mapping gene identifiers to canonical protein entries, structure identifiers, and metadata including resolution, coverage, and organism source.

In some embodiments, the data acquisition module 202 is further configured to manage compound data corresponding to known or candidate drugs. The data acquisition module 202 may access compound libraries or chemical databases such as ChEMBL, PubChem, or proprietary datasets, retrieving compound structures in formats such as SDF or MOL2. The compound data may include molecular weight, charge, atom types, 3D coordinates, and other descriptors required for downstream docking computations.

The data acquisition module 202 may also include preprocessing logic to filter or validate incoming data. For example, the data acquisition module 202 may verify that each protein structure file corresponds to a human gene, remove entries lacking atomic coordinates, and exclude incomplete or low-resolution structures below a predetermined threshold. In some embodiments, the data acquisition module 202 may compute sequence alignment statistics to identify homologous or redundant entries, ensuring that only representative structures are passed to the structure selection module 204.

In another embodiment, the data acquisition module 202 maintains a local cache or database to store retrieved information for reuse and faster access. The local cache may include indexing and version control capabilities to track database updates or retractions, allowing the target validation apparatus 104 to operate both online and offline. The data acquisition module 202 may further include a scheduling component for periodic synchronization with external databases, ensuring that the system continuously operates with the most current structural and sequence information available.

Through these operations, the data acquisition module 202 provides the foundational dataset from which all downstream modules operate, ensuring that the system begins with accurate, comprehensive, and standardized biological and chemical inputs necessary for large-scale computational target validation.

In one embodiment, the structure selection module 204 is configured to evaluate, rank, and select optimal protein structures or structure fragments for each gene based on sequence coverage, structural resolution, and data quality metrics. The structure selection module 204 receives as input a plurality of protein structure files retrieved and standardized by the data acquisition module 202. These input structures may correspond to different regions or conformations of a protein encoded by a single gene, such as full-length proteins, catalytic domains, or binding fragments derived from experimental studies.

The structure selection module 204, in one embodiment, analyzes metadata associated with each structure file, including parameters such as resolution (Ångströms), sequence coverage (percent of the canonical amino acid sequence represented), method of determination (e.g., X-ray crystallography, NMR, cryo-electron microscopy), and quality indicators such as R-factors or B-factors. The structure selection module 204 utilizes these metrics to compute a ranking or selection score that represents the suitability of each structure for docking analysis.

In one embodiment, the structure selection module 204 executes a coverage-resolution selection algorithm, which evaluates structure files according to a set of ordered ranges or intervals along the protein sequence. The algorithm may sort available structures by start and end positions on the protein sequence, prioritize higher coverage and higher-resolution structures, and apply overlap resolution rules to eliminate redundant or low-quality fragments. For example, the structure selection module 204 may select a structure if it does not overlap with a previously selected range or replace an overlapping structure if the new structure offers improved resolution or coverage. The selection results in a non-overlapping set of representative structures that, when combined, maximize sequence coverage for the gene while maintaining optimal data quality.

The structure selection module 204 may further incorporate heuristic or statistical models to refine its selection process. In certain embodiments, the structure selection module 204 applies machine learning techniques trained on historical docking outcomes to predict which structural fragments are most informative for docking analysis. In other embodiments, the structure selection module 204 includes logic to adjust for experimental uncertainty by weighting structures according to resolution variance or sequence homology to reference models.

Once the final structure set is selected, the structure selection module 204 outputs a curated list of structure identifiers and corresponding coordinate files to the structure preparation module 206. The output data may include metadata such as file source, chain identifier, sequence boundaries, and ranking score. The structure selection module 204 may also generate diagnostic logs or visual reports summarizing coverage distribution across each protein sequence, enabling validation of structure completeness prior to docking.

In some embodiments, the structure selection module 204 may cache intermediate selection results to enable incremental updates when new structure data become available. The structure selection module 204 may store version identifiers or timestamps for each selected structure, allowing reproducibility and traceability across different docking campaigns. By combining quantitative scoring, algorithmic filtering, and metadata validation, the structure selection module 204 ensures that only the most representative, high-quality protein structures are advanced to the structure preparation module 206 for downstream simulation.

In one embodiment, the structure preparation module 206 is configured to clean, standardize, and preprocess the selected protein structure files received from the structure selection module 204 to generate docking-ready molecular models. The structure preparation module 206 ensures that all protein structures used in the subsequent docking simulations are chemically complete, structurally consistent, and compatible with the input requirements of the docking computation module 208.

The structure preparation module 206, in one embodiment, removes extraneous molecular components and artifacts from each structure file that are unrelated to the target gene or that could interfere with docking accuracy. Such components may include water molecules, heteroatoms, buffer ions, cofactors, and non-native ligands that are not directly involved in the binding site of interest. The structure preparation module 206 may further remove alternate side-chain conformations, truncate incomplete residues, and resolve missing backbone atoms through reconstruction or homology-based modeling.

Following the cleaning step, the structure preparation module 206 performs chemical normalization and protonation of the protein structure. This includes the addition of hydrogen atoms according to a user-defined or automatically determined pH value (typically physiological pH ˜7.0). The structure preparation module 206 may calculate partial charges using empirical or quantum-mechanical methods, assign atom types compatible with a docking force field (such as AutoDock, AMBER, or CHARMM parameter sets), and validate bond orders and valency. In certain embodiments, the structure preparation module 206 may employ automated tools or integrated subroutines for protonation and charge assignment, such as PDB2PQR, OpenBabel, or in-house preprocessing scripts.

In another embodiment, the structure preparation module 206 is configured to define or refine active-site regions or binding pockets for docking. The structure preparation module 206 may identify binding sites using known ligand positions, pocket-detection algorithms, or sequence homology to proteins with characterized active sites. Alternatively, the structure preparation module 206 may generate multiple docking grids across the surface of the protein to perform unbiased binding-site screening. The structure preparation module 206 may store the resulting grid coordinates, pocket identifiers, and associated metadata in a format compatible with the docking computation module 208.

The structure preparation module 206 may further perform energy minimization or local relaxation of the protein structure to relieve steric clashes and improve geometrical consistency prior to docking. This process may use a limited number of optimization steps under a selected force field and cutoff criteria to maintain overall protein conformation while optimizing side-chain positioning in the binding region. The resulting structure files may be validated using quality metrics such as RMSD (root-mean-square deviation) and bond-angle distribution to ensure convergence and physical plausibility.

Once preprocessing is complete, the structure preparation module 206 outputs the cleaned and standardized protein structure files, along with associated grid or pocket data, to the docking computation module 208. The output may include file identifiers, parameter files, and metadata describing preparation settings such as pH, protonation state, and minimization parameters. In some embodiments, the structure preparation module 206 also maintains a preparation log recording all modifications performed on each structure, ensuring reproducibility and traceability across successive docking runs.

Through these operations, the structure preparation module 206 provides high-quality, chemically consistent, and structurally validated protein models that form the computational foundation for accurate and reproducible docking simulations within the target validation apparatus 104.

In one embodiment, the docking computation module 208 is configured to perform molecular docking simulations between a plurality of compounds and the protein structures prepared by the structure preparation module 206. The docking computation module 208 serves as the core computational engine of the target validation apparatus 104, responsible for simulating and characterizing potential interactions between candidate molecules and target proteins associated with the genes under analysis.

The docking computation module 208, in one embodiment, receives as input a set of docking-ready protein structures, binding-site definitions, and compound structure files. The docking computation module 208 converts these inputs into compatible formats for a docking engine or other computational chemistry software. The docking computation module 208 may use standard molecular docking programs, such as AutoDock Vina, Glide, GOLD, or proprietary algorithms, and may be configured to specify docking parameters including exhaustiveness, grid resolution, search mode, and the number of output binding poses. The docking computation module 208 can perform either rigid docking, where the protein conformation is fixed, or flexible docking, where side-chain or ligand flexibility is considered.

In some embodiments, the docking computation module 208 executes docking tasks in parallel across multiple processors, cores, or distributed computing environments. The docking computation module 208 may manage task distribution, job scheduling, and resource allocation using a parallelization framework such as MPI (Message Passing Interface), CUDA, or cloud orchestration systems. The docking computation module 208 may also communicate with external computing clusters or servers 108 through the data network 106 to submit, monitor, and retrieve distributed docking results. Each docking job may correspond to a specific compound-gene pair, allowing the system to analyze thousands of protein-ligand interactions concurrently.

The docking computation module 208 may further include a scoring and evaluation subcomponent that calculates preliminary binding energies or docking scores during simulation. This subcomponent may use scoring functions based on empirical, force-field, or machine-learning-derived models that estimate the binding affinity between the ligand and the protein binding site. The docking computation module 208 can generate multiple predicted binding poses per compound and store both the docking coordinates and the corresponding scores for downstream analysis by the scoring module 210.

In certain embodiments, the docking computation module 208 is configured to apply post-processing filters to identify valid or converged docking results. The docking computation module 208 may discard docking attempts that fail to meet predefined criteria, such as energy thresholds, structural completeness, or convergence quality. The docking computation module 208 may also perform re-scoring or pose clustering to consolidate similar binding conformations and to ensure that the best-scoring pose is selected for each compound-protein pair.

The docking computation module 208 outputs its results in structured data form, typically as a collection of docking scores, compound identifiers, and corresponding protein structure identifiers. The output data are transmitted to the scoring module 210 for aggregation, normalization, and further statistical analysis. In some embodiments, the docking computation module 208 maintains a computation log containing job identifiers, parameter settings, resource utilization metrics, and timestamps to ensure full traceability of each docking operation.

Through these functions, the docking computation module 208 provides a scalable, high-throughput framework for simulating protein-ligand interactions, enabling the target validation apparatus 104 to evaluate binding affinities across large sets of compounds and genes with reproducibility and computational efficiency.

In one embodiment, the scoring module 210 is configured to analyze, normalize, and aggregate the docking results generated by the docking computation module 208 to determine quantitative binding affinities between each of the plurality of compounds and the one or more protein structures. The scoring module 210 receives as input a dataset containing docking scores, predicted binding poses, and associated metadata such as compound identifiers, protein structure identifiers, and energy values calculated during docking simulations. The scoring module 210 processes this information to derive standardized affinity metrics suitable for downstream target validation and ranking.

The scoring module 210, in one embodiment, implements one or more scoring functions or statistical models that transform docking energy outputs into comparable affinity measures. The scoring module 210 may use empirical or semi-empirical scoring functions based on van der Waals interactions, electrostatics, hydrogen bonding, hydrophobic effects, and desolvation energies. Alternatively, the scoring module 210 may employ knowledge-based or machine-learning-based scoring methods that have been trained on experimentally determined protein-ligand complexes. The scoring module 210 may normalize energy values across docking runs performed under different parameter settings to produce dimensionless binding scores on a common scale.

In certain embodiments, the scoring module 210 is configured to select, for each gene or protein structure, the lowest or most favorable docking score corresponding to the best predicted binding pose of each compound. The scoring module 210 may apply additional filtering logic to remove outliers or docking results that fail to meet defined confidence thresholds. For example, the scoring module 210 may exclude results with docking energies above a specified cutoff, poses with excessive steric clashes, or simulations with incomplete convergence. The scoring module 210 may also perform clustering or statistical averaging of multiple binding poses to generate consensus binding affinities that better reflect the stability of compound-protein interactions.

The scoring module 210 may further incorporate quality-control routines to ensure reproducibility and consistency across large datasets. These routines may include verifying that each gene and compound pair has at least one valid docking entry, detecting missing or corrupted files, and recording provenance metadata linking each score to the original docking job executed by the docking computation module 208. In some embodiments, the scoring module 210 is configured to compute derived statistical parameters such as mean binding energy, standard deviation, or percentile ranks for each target to facilitate subsequent comparative analysis.

Once the binding affinities have been determined, the scoring module 210 aggregates the results into a unified data structure that maps compound identifiers to corresponding gene or protein targets. This aggregated dataset may include, for each compound-target pair, the best docking score, associated structure identifier, and quality metrics. The scoring module 210 outputs this dataset to the target analysis module 212 for ranking, filtering, and target validation.

In some embodiments, the scoring module 210 may perform intermediate visualization or reporting functions, such as generating histograms, scatter plots, or summary tables of docking performance across compounds or genes. The scoring module 210 may also log parameter settings, scoring functions used, and software versions to support reproducibility and regulatory compliance. Through these operations, the scoring module 210 converts raw docking results into standardized, interpretable affinity data that serve as the quantitative foundation for determining validated and novel targets within the target validation apparatus 104.

In one embodiment, the target analysis module 212 is configured to process, interpret, and evaluate the aggregated binding affinity data received from the scoring module 210 to generate a list of validated or novel biological targets. The target analysis module 212 serves as the interpretive and decision-making component of the target validation apparatus 104, transforming quantitative affinity data into biologically meaningful conclusions about compound-target relationships.

The target analysis module 212, in one embodiment, receives as input a dataset mapping each compound to one or more protein targets, along with their respective binding affinities and quality metrics. The target analysis module 212 analyzes these data to identify targets that demonstrate significant binding affinity to specific compounds or classes of compounds. The target analysis module 212 may employ threshold-based filtering, statistical ranking, or multi-criteria evaluation to classify targets as validated, novel, or non-relevant. For example, the target analysis module 212 may designate a target as validated if its computed binding affinity exceeds a predetermined threshold or falls within the top percentile of results for a given compound.

In certain embodiments, the target analysis module 212 is configured to detect and exclude promiscuous targets, defined as genes or proteins that appear among the top-ranked results for multiple compounds or exhibit binding affinities across a wide range of structurally unrelated ligands. The target analysis module 212 may implement frequency-based filters, statistical enrichment tests, or clustering methods to identify and remove such promiscuous targets from the ranked list. This filtering step enhances the specificity of the validation process by focusing on biologically meaningful and compound-selective interactions.

The target analysis module 212 may also incorporate machine-learning or artificial-intelligence components to improve predictive accuracy. In one embodiment, the target analysis module 212 applies a trained machine-learning model that receives docking-derived affinity data, compound descriptors, and protein features as input and outputs predictions regarding off-target interactions, toxicity risks, or likelihood of biological relevance. The machine-learning model may be trained using experimental binding data, toxicity assays, or historical docking results to enhance its predictive capability. The target analysis module 212 may update or retrain the model iteratively as new data are acquired, allowing continuous improvement of prediction accuracy.

In another embodiment, the target analysis module 212 generates confidence scores or ranking indices for each target. These scores may combine factors such as binding energy, docking convergence, structural resolution, and prior biological evidence. The target analysis module 212 may assign higher confidence to targets with consistent high-affinity results across multiple structures or compounds, while down-weighting targets associated with variable or uncertain results. The target analysis module 212 may also compute correlation or network metrics to identify potential target families, pathways, or off-target clusters.

The output of the target analysis module 212 is a structured list or database of validated or novel targets. Each entry in the output may include a gene identifier, protein name, compound identifier, best docking score, confidence score, and any machine-learning-derived annotations. The target analysis module 212 transmits this output to the results management module 214 for storage, visualization, or export.

In some embodiments, the target analysis module 212 supports user-defined analysis modes, such as drug-repurposing analysis, toxicity mapping, or cross-target comparison. The target analysis module 212 may include user-configurable parameters for adjusting binding-affinity thresholds, filtering criteria, or machine-learning model selection. Through these capabilities, the target analysis module 212 transforms raw docking data into actionable insight, enabling the target validation apparatus 104 to prioritize targets for further biological validation, clinical study, or compound optimization.

In one embodiment, the results management module 214 is configured to organize, store, visualize, and export the results generated by the target analysis module 212. The results management module 214 serves as the data integration and output component of the target validation apparatus 104, ensuring that validated and novel targets, along with their associated metadata, are maintained in a structured and accessible format for subsequent review, reporting, or external processing.

The results management module 214, in one embodiment, receives as input a dataset containing ranked or filtered targets identified by the target analysis module 212. This dataset may include gene identifiers, protein names, compound identifiers, binding-affinity values, confidence scores, and any annotations generated by machine-learning or predictive models. The results management module 214 organizes this data into relational tables or graph-based data structures that link compounds to corresponding targets and binding metrics. The results management module 214 may store the organized data in a database management system or non-volatile storage medium that supports indexing, querying, and cross-referencing of compound-target relationships.

In some embodiments, the results management module 214 is configured to output results in one or more file formats suitable for downstream use, such as comma-separated value (CSV) files, JavaScript Object Notation (JSON), Extensible Markup Language (XML), or specialized bioinformatics data formats. The results management module 214 may further include export capabilities that allow the target validation apparatus 104 to transmit data to remote servers 108, laboratory information systems, or external drug discovery platforms via the data network 106. The results management module 214 may also provide an API that enables automated access to the validated target data for integration with other analytical tools or visualization systems.

The results management module 214 may include data visualization and reporting components that enable users or downstream systems to interpret and analyze results interactively. These components may generate ranked lists, heat maps, or network diagrams illustrating compound-target affinity relationships, promiscuity patterns, or confidence distributions. In certain embodiments, the results management module 214 provides a dashboard interface that allows users to filter results by affinity threshold, compound class, or gene family, and to export customized subsets of the data for experimental validation or publication.

To maintain data integrity and traceability, the results management module 214 may record metadata associated with each result set, including processing parameters, algorithm versions, timestamps, and user identifiers. This information may be used to ensure reproducibility of analyses and to support auditing or regulatory documentation. The results management module 214 may also include version-control capabilities that allow storage of multiple analysis runs for comparison or rollback, ensuring that prior results are preserved for reference or validation purposes.

In another embodiment, the results management module 214 implements data security and access control mechanisms. The results management module 214 may support role-based permissions, encrypted storage, and authenticated data transmission to ensure that sensitive biological and compound data are protected from unauthorized access. The results management module 214 may further perform periodic backups to local or remote repositories to prevent data loss and ensure business continuity.

Through these operations, the results management module 214 provides a robust and flexible framework for managing the outputs of the computational target identification and validation process. By maintaining organized, queryable, and exportable records of validated and novel targets, the results management module 214 enables efficient interpretation, reproducibility, and downstream application of the results generated by the target validation apparatus 104.

In one embodiment, the AI module 216 is configured to perform artificial intelligence and machine-learning operations that support, enhance, and continuously improve the computational processes of the target validation apparatus 104. The AI module 216 may be trained to predict compound-target interactions, off-target binding events, or toxicity profiles based on data generated by the other modules 202 through 214. The AI module 216 may also refine its predictive models over time using validated experimental results, updated docking outcomes, or additional biological information retrieved by the data acquisition module 202.

The AI module 216, in one embodiment, receives as input aggregated datasets containing docking scores, binding affinities, compound descriptors, protein structure features, and prior prediction outcomes. The AI module 216 processes these data to identify statistical or nonlinear relationships between compound properties and binding outcomes, enabling improved ranking and filtering of targets within the target analysis module 212. The AI module 216 may employ one or more learning paradigms, including supervised, unsupervised, reinforcement, or transfer learning, depending on data availability and analysis objectives.

The AI module 216 may use regression, classification, or generative models such as random forests, support vector machines, graph neural networks, or transformer-based architectures to predict or model protein-ligand interactions. In certain embodiments, the AI module 216 performs feature extraction or dimensionality reduction on input data, generating standardized feature vectors that capture chemical, structural, and energetic properties of compounds and proteins. The AI module 216 may evaluate its models using cross-validation, loss minimization, or benchmark comparison, and select the best-performing models for deployment within the target analysis module 212.

In some embodiments, the AI module 216 operates in a closed feedback loop with the target analysis module 212 and the results management module 214. The AI module 216 may receive validated results or user feedback to update model parameters and retrain its predictive models automatically. The retrained models may then be used to adjust confidence scores, modify affinity thresholds, or reprioritize targets during subsequent analyses. The AI module 216 may further transmit learned parameters or updated models to the scoring module 210 to augment or replace conventional scoring functions with machine-learning-enhanced scoring strategies.

The AI module 216 may maintain a model repository containing trained models, training datasets, performance metrics, and version identifiers to ensure reproducibility and traceability of predictive outputs. The module may also perform model optimization or hyperparameter tuning to improve predictive accuracy and generalization performance. Through these operations, the AI module 216 enables adaptive learning and intelligent automation within the target validation apparatus 104, allowing the system to continuously refine its analytical precision and predictive capability as new data become available.

In one embodiment, the modules 202 through 216 of the target validation apparatus 104 are communicatively and functionally coupled to operate as an integrated and adaptive pipeline for computational target identification and validation. Each module performs a distinct stage of data processing, and the output of one module serves as the input for the next, establishing a continuous and automated workflow that may operate sequentially, in parallel, or in a hybrid configuration. Communication among the modules may occur through shared memory, high-speed data buses, message queues, or network-based communication protocols implemented within or across hardware and software components of the target validation apparatus 104.

During operation, the data acquisition module 202 retrieves and standardizes protein, gene, and compound data from external or local databases. The curated dataset is transmitted to the structure selection module 204, which identifies and ranks representative protein structures for each gene based on sequence coverage and resolution. The selected structures are then provided to the structure preparation module 206, which performs chemical and geometric preprocessing to produce docking-ready molecular models. The prepared structures and compound libraries are forwarded to the docking computation module 208, which performs large-scale docking simulations to evaluate potential compound-protein interactions.

The docking computation module 208 outputs docking results and energy scores to the scoring module 210, which processes the data to compute normalized binding affinities and statistical summaries. The scoring module 210 then transmits the processed affinity data to the target analysis module 212, which interprets the results, identifies validated and novel targets, and applies filtering logic to remove promiscuous or low-confidence targets. The target analysis module 212 generates ranked target lists and confidence scores, which are transferred to the results management module 214 for organization, visualization, and export in standardized formats.

In certain embodiments, the AI module 216 operates cooperatively with one or more of the other modules to enhance prediction accuracy and automate continuous improvement. The AI module 216 may receive input data and results from the scoring module 210, the target analysis module 212, or the results management module 214, and use this information to train, refine, or update machine-learning models that improve the system's predictive performance. The AI module 216 may output updated model parameters or predictive results back to the target analysis module 212 to adjust ranking criteria, to the scoring module 210 to augment scoring functions, or to the structure selection module 204 to guide future structure prioritization.

The AI module 216 may also participate in a feedback loop whereby validated experimental results or updated biological datasets trigger automatic retraining of predictive models, ensuring that the target validation apparatus 104 adapts to new data and maintains up-to-date performance. In certain embodiments, the AI module 216 manages a repository of trained models and metadata accessible by other modules to ensure reproducibility and version control of machine-learning workflows.

The modules 202 through 216 may be implemented within a single computing device or distributed across multiple servers 108 connected via the data network 106. The target validation apparatus 104 may coordinate module operations using distributed computing frameworks, job schedulers, or containerized microservices. Data exchange between modules may employ serialization protocols, application programming interfaces, or secure file transfers to ensure reliability and integrity of results.

Through this modular and adaptive architecture, the target validation apparatus 104 provides an end-to-end, scalable, and self-improving computational framework for identifying and validating biological targets. The inclusion of the AI module 216 allows the system to learn from prior outcomes, enhance prediction precision, and continuously evolve as new biological, chemical, or experimental data become available.

In one embodiment, the target validation apparatus 104 may be implemented as a large-scale or distributed computational system configured to execute docking, scoring, and analysis operations across multiple processing units, servers, or networked computing nodes. The distributed implementation enables parallel execution of docking simulations and affinity calculations for thousands of compound-gene pairs, significantly increasing computational throughput and reducing overall processing time. The target validation apparatus 104 may employ distributed computing frameworks, such as cloud-based orchestration systems, high-performance computing clusters, or containerized microservices, to coordinate workload allocation among instances of the docking computation module 208, the scoring module 210, and the target analysis module 212.

The target validation apparatus 104 may include a task scheduler or job management component configured to divide compound and structure datasets into smaller computational batches and assign those batches to available compute resources. Each distributed node may operate an independent instance of one or more modules, execute docking or scoring tasks locally, and return results to a centralized aggregation process for further analysis by the scoring module 210 or target analysis module 212. The system may synchronize intermediate results and maintain consistency through checkpointing, redundancy, and error-recovery mechanisms to ensure reliability during large-scale processing.

In certain embodiments, the target validation apparatus 104 may leverage elastic resource scaling, dynamically provisioning or deallocating computational nodes in response to workload demand. Communication among distributed nodes may occur through high-speed interconnects, message-passing interfaces, or secure network protocols that support efficient transfer of molecular data and docking results. The system may further employ distributed data storage architectures, such as parallel file systems or object stores, to enable simultaneous access to compound libraries, protein structure databases, and intermediate docking files.

The distributed configuration allows the target validation apparatus 104 to process expansive datasets that would otherwise exceed the capacity of a single computing device, making it suitable for enterprise-scale drug discovery, toxicity screening, and target validation pipelines. In certain embodiments, the distributed framework also enables federated or collaborative operation across institutional boundaries, allowing multiple research sites or data centers to contribute computing resources and validated results to a shared analytical environment. Through these scalable and networked implementations, the target validation apparatus 104 provides robust performance, high availability, and reproducible computational efficiency across a wide range of deployment configurations.

In one exemplary use case, the target validation apparatus 104 is employed within a pharmaceutical research facility to identify and validate potential biological targets for a known anticancer compound. The research team begins by providing the compound's chemical structure file, along with configuration parameters for docking precision, target organism (human), and acceptable resolution thresholds, to the data acquisition module 202. The data acquisition module 202 retrieves corresponding protein sequences and structure data for all human genes from curated biological databases such as UniProt and PDBe-KB, filtering out entries that lack atomic coordinates or fall below the desired resolution.

The curated protein data are transmitted to the structure selection module 204, which applies a coverage-resolution ranking algorithm to identify representative protein fragments for each gene. For example, if multiple PDB structures exist for a kinase family gene, the structure selection module 204 selects the fragment that maximizes sequence coverage while maintaining the highest available crystallographic resolution. These selected structures are then processed by the structure preparation module 206, which removes non-relevant ligands, adds hydrogen atoms at physiological pH, and performs a short energy minimization to eliminate steric clashes. The prepared protein structures are then passed to the docking computation module 208.

The docking computation module 208 executes molecular docking simulations between the anticancer compound and each prepared protein structure. The docking computation module 208 distributes the docking jobs across a network of available processing nodes, enabling thousands of docking tasks to be performed in parallel. Each docking task produces one or more binding poses and corresponding energy scores, which are transmitted to the scoring module 210. The scoring module 210 analyzes the docking scores, identifies the most favorable binding pose for each protein, and normalizes the results to produce standardized binding affinities across all genes.

The target analysis module 212 receives the aggregated binding data and determines which genes show the strongest predicted interaction with the compound. For instance, if multiple kinases display sub-micromolar predicted binding affinities, the module prioritizes those targets while filtering out promiscuous proteins that bind many unrelated compounds. The target analysis module 212 may also invoke the AI module 216 to compare the predicted binding patterns with prior experimental datasets. The AI module 216 may refine confidence scores or predict possible off-target effects, such as binding to cardiac ion channels associated with known toxicity risks.

The results management module 214 compiles the final ranked list of validated and novel targets, complete with compound identifiers, binding energies, and AI-generated confidence metrics. These results are stored in a local or cloud-based database and can be exported as a CSV file or visualized through a dashboard that highlights the top predicted protein interactions. The research team can then use these results to guide in vitro validation experiments or to explore potential drug-repurposing opportunities for other compounds within the same target families.

In another embodiment, the same system can be used for toxicity screening, where the compound list includes environmental chemicals or drug metabolites. In that context, the target validation apparatus 104 identifies which proteins are most likely to mediate adverse biological effects. In each case, the system automates the entire process—from data retrieval to docking, scoring, and interpretation—reducing the time and computational effort traditionally required for large-scale target discovery and validation studies.

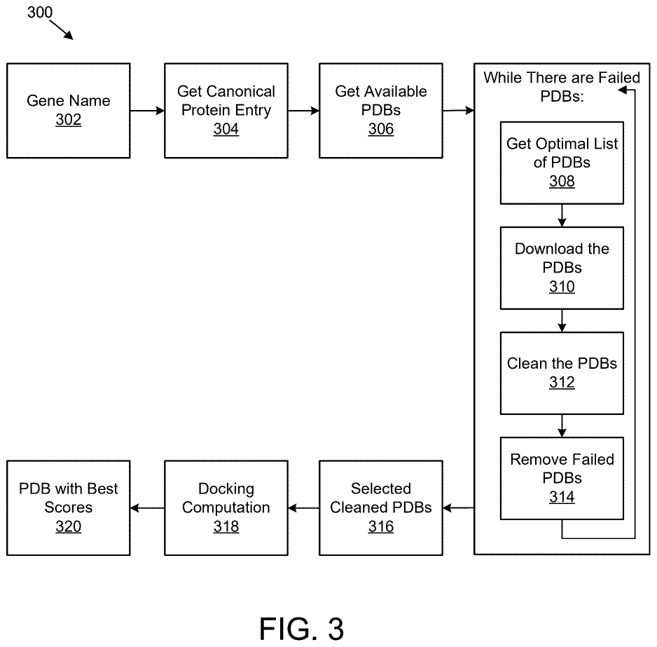

FIG. 3 illustrates one embodiment of a gene-based docking workflow 300 implemented by the target validation apparatus 104. In one embodiment, the workflow 300 begins at a gene identification block 302, in which a gene of interest is selected for analysis. The gene identification block 302 is performed by the data acquisition module 202, which accesses curated genomic or proteomic databases to identify genes associated with the desired organism or disease state.

Next, a protein entry retrieval block 304, also executed by the data acquisition module 202, obtains the canonical protein entry corresponding to the selected gene from sources such as UniProt or PDBe-KB. The structure retrieval block 306, likewise implemented by the data acquisition module 202, gathers all available protein structure files associated with the gene, including multiple PDB entries (for example, PDB 1, PDB 2, PDB 3).

The structure selection block 308, performed by the structure selection module 204, applies a coverage-resolution ranking algorithm to evaluate the available structures and to select an optimal subset based on sequence coverage, resolution, and quality metrics. The structure download block 310, also coordinated by the structure selection module 204, retrieves the chosen PDB files and forwards them to the structure preparation module 206.

The structure cleaning block 312, executed by the structure preparation module 206, preprocesses each selected protein structure by removing water molecules, heteroatoms, and side chains unrelated to the target gene and by adding hydrogen atoms or charge assignments based on a predetermined pH value. Invalid or incomplete structures are filtered at a failed-structure removal block 314, also performed by the structure preparation module 206, to ensure that only validated and complete models proceed to docking.

The prepared-structure block 316 represents the output stage of the structure preparation module 206, in which the selected protein structure files have been cleaned, validated, and standardized for docking. At the prepared-structure block 316, the system produces a finalized set of selected cleaned PDBs—protein data bank files that have been filtered to remove incomplete or low-quality entries, stripped of non-relevant molecules such as water or heteroatoms, and supplemented with hydrogen atoms and charge assignments based on a predetermined pH. Each cleaned PDB file corresponds to a protein structure that meets the sequence-coverage and resolution thresholds established by the structure selection module 204. The prepared-structure block 316 outputs these docking-ready protein models, together with their associated metadata, to the docking computation block 318 for execution of molecular docking simulations by the docking computation module 208.

The docking computation block 318 receives as input the selected and cleaned PDB files generated by the structure preparation module 206 and performs molecular docking simulations between the compound of interest and each prepared protein structure. The docking computation block 318 is executed by the docking computation module 208, which may employ a molecular docking engine configured for high exhaustiveness and multiple output modes to predict compound-protein binding poses and associated energy scores. In one embodiment, the docking computation module 208 utilizes AutoDock Vina, an open-source molecular docking program that estimates ligand- protein binding conformations and energies, or any equivalent docking engine capable of performing similar calculations. The docking computation block 318 produces a set of predicted binding configurations and corresponding docking scores representing the estimated binding affinities between the compound and each candidate protein structure.

The best-score selection block 320, performed by the scoring module 210 in cooperation with the results management module 214, evaluates the docking results generated by the docking computation block 318 and identifies, for each gene, the protein structure that yields the lowest or most favorable docking score. The best-score selection block 320 ranks the docking outcomes by predicted binding energy, filters invalid or incomplete entries, and designates the top-scoring protein structure as the PDB with best scores for that gene. The resulting data, including the selected PDB identifier, compound identifier, and associated binding-affinity value, are recorded and transmitted to downstream processes for aggregation and large-scale target validation by other modules of the target validation apparatus 104.

The output generated by the PDB-with-best-scores block 320 may include a gene identifier, PDB accession code, compound identifier, and computed binding-affinity value. The information produced by the workflow 300 for each gene serves as input to subsequent large-scale docking and aggregation processes performed by the other modules of the target validation apparatus 104, including the target analysis module 212 and the AI module 216, which together refine, interpret, and validate the compiled docking results.

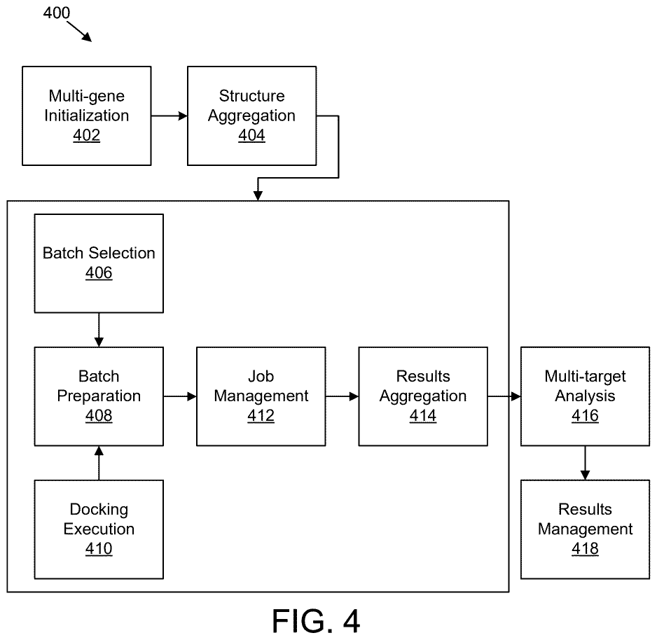

FIG. 4 illustrates one embodiment of a large-scale docking workflow 400 implemented by the target validation apparatus 104 to perform parallelized target identification and validation across multiple genes and compounds. The workflow 400 expands the single-gene docking process of FIG. 3 to operate concurrently on a genome-or compound-library scale.

The workflow 400 begins at a multi-gene initialization block 402, executed by the data acquisition module 202, which compiles a list of genes, their associated canonical protein entries, and compound identifiers designated for screening. The structure aggregation block 404, also performed by the data acquisition module 202, retrieves and consolidates all structure data corresponding to the selected genes from one or more biological databases. The batch selection block 406, implemented by the structure selection module 204, evaluates the available structures for all genes using the coverage-resolution ranking algorithm and partitions them into optimized structure batches for downstream processing.

The batch preparation block 408, carried out by the structure preparation module 206, cleans and standardizes all selected structures across the dataset, ensuring chemical completeness and consistency in protonation and charge states. The cleaned structure batches are transmitted to a parallel docking execution block 410, executed by the docking computation module 208, which distributes molecular docking tasks across multiple processors, servers 108, or networked computing nodes. The docking computation module 208 executes a docking engine such as AutoDock Vina in high-exhaustiveness and high-mode configurations, generating docking scores for every compound-gene pair.

A job management block 412, implemented by the scoring module 210 in coordination with distributed computing frameworks, monitors the progress of all docking tasks, handles job scheduling, and collects partial results. The intermediate docking outputs are transferred to a results aggregation block 414, also managed by the scoring module 210, which consolidates the docking scores and extracts the lowest (best) binding-energy values for each gene-compound combination.

The aggregated dataset is analyzed at a multi-target analysis block 416, executed by the target analysis module 212, which identifies validated and novel targets across the entire dataset, filters promiscuous genes that appear among top-ranked results for multiple compounds, and assigns confidence levels or statistical weights to each remaining target. The AI module 216 may operate cooperatively with the target analysis module 212 at this stage to refine confidence scores, predict off-target interactions, or update trained models based on aggregated docking data.

The filtered and ranked results are then transmitted to a large-scale results management block 418, performed by the results management module 214, which compiles the final dataset into an exportable format, such as a comma-separated-value (CSV) file, database table, or graph-structured repository linking compounds to their corresponding targets. The results management module 214 may also interface with external analytical or visualization systems to display the ranked target lists or to enable further downstream processing.

Through these operations, the workflow 400 enables simultaneous docking, scoring, and validation of thousands of gene-compound pairs. The integration of the modules 202 through 216 allows the target validation apparatus 104 to execute large-scale docking campaigns efficiently, maintain data integrity across distributed computing resources, and continuously improve predictive accuracy through AI-assisted analysis and model refinement.

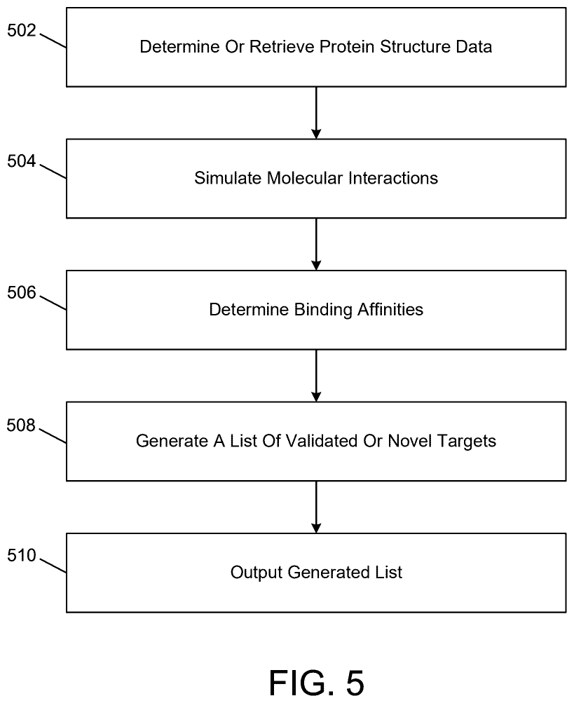

FIG. 5 depicts one embodiment of a method for computational target identification and validation. In one embodiment, the method may be performed by an information handling device 102, the target validation apparatus 104, or one or more modules of the apparatus, including the data acquisition module 202, the docking computation module 208, the scoring module 210, the target analysis module 212, and the results management module 214. The method may be implemented as computer-executable instructions stored in a non-transitory computer-readable medium and executed by one or more processors.

The method begins at a data determination step 502, in which the system determines or retrieves protein structure data associated with a plurality of genes. The data determination step 502 may include accessing curated biological databases, such as UniProt and PDBe-KB, to obtain canonical protein entries and associated three-dimensional structure files. The retrieved data describe one or more protein structures that are prepared for computational analysis by the data acquisition module 202.

At a simulation step 504, the method simulates molecular interactions between a plurality of compounds and the one or more protein structures. The simulation step 504 may be executed by the docking computation module 208, which performs docking simulations using a molecular-docking engine configured with high exhaustiveness and multiple output modes. Each simulation produces predicted binding poses and preliminary binding-energy values.

At a binding-affinity determination step 506, the method determines binding affinities between each of the plurality of compounds and the one or more protein structures. The binding-affinity determination step 506 may be carried out by the scoring module 210, which processes docking results, calculates standardized affinity scores, and identifies the most favorable binding configuration for each compound-protein pair.

The method proceeds to a target-generation step 508, in which the method generates a list of validated or novel targets based on the determined binding affinities. The target-generation step 508 may be performed by the target analysis module 212 in cooperation with the results management module 214. This step may include ranking targets by binding strength, filtering promiscuous or low-confidence results, and storing or exporting the final ranked list of validated and novel targets for further analysis.

The method terminates at an end step 510, where the generated list of targets is output, stored, or transmitted for downstream drug-discovery, repurposing, or toxicology workflows.

In some embodiments, the method may be executed iteratively or automatically refined by the AI module 216 of the target validation apparatus 104. During or after completion of the target-generation step 508, the AI module 216 may analyze the resulting binding-affinity data, target rankings, and any available experimental validation results to assess prediction accuracy. The AI module 216 may identify systematic biases, underrepresented target classes, or inconsistencies among docking scores and use this information to retrain or update one or more machine-learning models. The updated models may then adjust parameters of the simulation step 504, such as docking exhaustiveness, grid size, or scoring weights, or modify the affinity-determination criteria used in step 506 to improve precision and reproducibility.

The AI module 216 may further incorporate new compound libraries, protein structures, or validated binding data retrieved by the data acquisition module 202 to expand the scope of the analysis. Through these feedback operations, the AI module 216 enables the method to operate as a self-improving computational process that continuously refines its predictive models and target-ranking algorithms. This adaptive framework allows the system to maintain high predictive accuracy as biological databases evolve, new compounds are introduced, and experimental results become available, thereby supporting ongoing discovery, repurposing, and toxicity-assessment activities within the same computational environment.

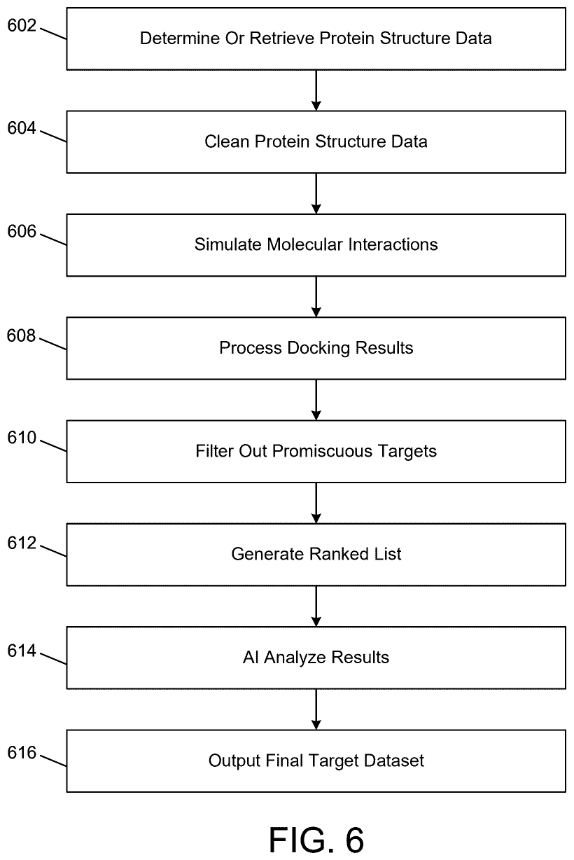

FIG. 6 depicts one embodiment of an enhanced method for computational target identification and validation. In one embodiment, the method may be performed by an information-handling device, the target validation apparatus 104, or one or more of its modules, including the data acquisition module 202, the structure preparation module 206, the docking computation module 208, the scoring module 210, the target analysis module 212, the results management module 214, and the AI module 216. The method extends the method of FIG. 5 by incorporating additional preprocessing, filtering, and adaptive-learning steps to further refine the accuracy and reliability of the computational workflow.

The method begins at a data determination step 602, in which the system determines or retrieves protein structure data associated with a plurality of genes, as previously described. The data determination step 602 may be performed by the data acquisition module 202, which accesses canonical protein entries and related structure files from curated databases.

A data-preprocessing step 604 follows, executed by the structure preparation module 206, in which the retrieved protein structures are cleaned and chemically standardized. This step may include removing water molecules, adding hydrogen atoms based on pH, and ensuring geometric consistency of the structures before docking.

At a simulation step 606, the docking computation module 208 simulates molecular interactions between a plurality of compounds and the prepared protein structures using a high-exhaustiveness docking configuration. Each simulation produces one or more predicted binding poses and preliminary docking scores.

At a binding-affinity determination step 608, the scoring module 210 processes the docking results and computes standardized binding-affinity values for each compound-protein pair.

A target-filtering step 610 then occurs, performed by the target analysis module 212, where the system filters out promiscuous or low-confidence targets that appear among the top results for multiple compounds. This step enhances the specificity of subsequent validation and ranking operations.

The method continues with a target-generation step 612, executed by the target analysis module 212 in cooperation with the results management module 214, to generate a ranked list of validated or novel targets based on the binding affinities that remain after filtering.

An AI-based refinement step 614 may follow, implemented by the AI module 216. In this step, machine-learning models analyze the generated results to predict off-target interactions, assess toxicity risks, or re-rank targets using adaptive weighting algorithms. The AI module 216 may update internal models based on these findings to improve performance in subsequent iterations of the method.

Finally, the method concludes at an output step 616, performed by the results management module 214, which stores, visualizes, or exports the final validated-target dataset for use in downstream discovery or validation workflows.

In certain embodiments, the method may be executed iteratively or distributed across multiple computing resources to enhance computational efficiency and predictive accuracy. The target validation apparatus 104 may partition the dataset of compounds and genes into smaller computational batches and distribute them to parallel instances of the docking computation module 208 operating on different processors, servers 108, or nodes connected via the data network 106. Each distributed node may execute the data-preprocessing step 604, the simulation step 606, and the binding-affinity determination step 608 independently and transmit intermediate results to a centralized scoring or aggregation process managed by the scoring module 210.

The AI module 216 may monitor these distributed executions and adaptively modify parameters for each batch based on observed performance or data quality, enabling dynamic optimization of docking exhaustiveness, scoring thresholds, or filtering criteria in real time. The AI module 216 may also use validated outcomes from previous iterations to retrain predictive models and refine the target-filtering step 610 and the AI-based refinement step 614, allowing the method to improve continuously as new results are generated.

In some embodiments, the method operates in an asynchronous mode, where updated models or refined scoring parameters are automatically propagated to active docking tasks without interrupting execution. The results management module 214 may aggregate outputs from distributed nodes, reconcile duplicate entries, and update the ranked target dataset generated during the target-generation step 612. Through this iterative and distributed execution framework, the method achieves high throughput, adaptive learning, and scalable performance across large compound libraries and genome-wide protein datasets, providing reproducible and continuously improving results in computational target identification and validation.

As used herein, the following terms shall have the meanings set forth below unless the context clearly indicates otherwise. The definitions provided are intended to clarify the terminology used throughout this specification and the appended claims and are not intended to limit the scope of the invention.

The term “target validation” refers to computational or experimental processes used to confirm that a biological macromolecule, such as a protein, enzyme, or gene product, is functionally associated with a disease or phenotype of interest, and that modulation of the target is expected to produce a measurable biological or therapeutic effect. Target validation, as used in this disclosure, includes in silico validation performed by the target validation apparatus 104 through docking, scoring, and ranking operations.

The term “docking” refers to a computational process that predicts the preferred orientation, conformation, or binding mode of a ligand or compound when bound to a protein or other macromolecular structure. Docking may be performed using rigid-body or flexible algorithms and produces quantitative measures of binding strength or energy that are used to infer likely compound-target interactions.

The term “binding affinity” refers to a quantitative or semi-quantitative measure of the strength of interaction between a ligand or compound and a protein structure. Binding affinity may be expressed as an energy value, docking score, or any normalized metric that reflects the relative stability or favorability of the interaction. In the context of this disclosure, binding affinities are computationally determined by the scoring module 210.

The term “promiscuous target” refers to a gene or protein that demonstrates significant predicted or measured binding affinity to multiple structurally unrelated compounds, indicating non-specific or multi-target binding behavior. Promiscuous targets are typically identified and filtered out by the target analysis module 212 to improve the specificity of validated target predictions.

The term “coverage-resolution ranking algorithm” refers to a computational method used by the structure selection module 204 to evaluate a plurality of protein structures based on the portion of the amino acid sequence represented (coverage) and the experimental or computational resolution of the structure. The algorithm produces a ranked subset of structures that optimizes both completeness of sequence representation and data quality for downstream docking simulations.

The term “gene-based docking” refers to a computational workflow in which molecular docking simulations are performed for one or more compounds across protein structures corresponding to a plurality of genes, allowing gene-level assessment of compound-target interactions. Gene-based docking enables identification of both validated and novel targets within a genome-scale screening campaign.

The term “compound” refers to a small molecule, peptide, nucleic acid, or other chemical entity capable of binding to a protein or other macromolecular target, whether naturally occurring or synthetically derived. Compounds may include known drugs, experimental candidates, metabolites, or toxic substances used in repurposing, discovery, or toxicological assessment workflows.

In one embodiment, an apparatus is configured to determine protein structure data associated with a plurality of genes, wherein the protein structure data describes one or more protein structures; simulate interactions between a plurality of compounds and the one or more protein structures; determine binding affinities between each of the plurality of compounds and the one or more protein structures; and generate a list of validated or novel targets based on the binding affinities.

In one embodiment, determining the protein structure data comprises retrieving protein sequence and structure files from a biological database. In one embodiment, the apparatus is configured to select the structure files based on at least one of sequence coverage or resolution of available protein data.

In one embodiment, determining the protein structure data comprises cleaning the structure files by removing water, heterogens, and side chains unrelated to the protein, and adding hydrogen atoms based on a predetermined pH value. In one embodiment, determining the protein structure data comprises selecting structure fragments for each of the plurality of genes using a coverage-resolution ranking algorithm.

In one embodiment, simulating interactions comprises performing molecular docking using a docking engine configured with exhaustiveness and mode parameters. In one embodiment, simulating interactions comprises executing molecular docking computations in parallel across multiple processing units or servers.

In one embodiment, the apparatus is configured to aggregate docking scores from parallel docking simulations into a results file. In one embodiment, determining binding affinities comprises selecting, for each gene of the plurality of genes, a lowest docking score representing a most favorable binding configuration.

In one embodiment, the apparatus is configured to identify promiscuous targets based on a frequency of occurrence among top-ranked results for multiple compounds. In one embodiment, the apparatus is configured to exclude promiscuous targets that appear among top-ranked targets for more than half of the plurality of compounds.

In one embodiment, the apparatus is configured to assign a statistical confidence score to each of the validated or novel targets based on variance among corresponding docking results. In one embodiment, the apparatus is configured to apply a trained machine learning model configured to receive docking affinity data and output predicted off-target or toxic interactions based on the binding affinities.

In one embodiment, the list of validated or novel targets comprises gene identifiers, compound identifiers, and binding affinity scores for each of the validated or novel targets. In one embodiment, the apparatus is configured to store the list of validated or novel targets in a graph database linking compound identifiers to gene names and affinity metrics.

In one embodiment, a method includes determining protein structure data associated with a plurality of genes, wherein the protein structure data describes one or more protein structures, simulating interactions between a plurality of compounds and the one or more protein structures, determining binding affinities between each of the plurality of compounds and the one or more protein structures, and generating a list of validated or novel targets based on the binding affinities.

In one embodiment, the method includes determining the protein structure data comprises retrieving protein sequence and structure files from a biological database. In one embodiment, the method includes selecting the structure files based on at least one of sequence coverage or resolution of available protein data. In one embodiment, determining the protein structure data comprises cleaning the structure files by removing water, heterogens, and side chains unrelated to the protein, and adding hydrogen atoms based on a predetermined pH value.

In one embodiment, a computer program product comprises a non-transitory computer-readable medium storing instructions that, when executed by one or more processors, cause the one or more processors to determine protein structure data associated with a plurality of genes, wherein the protein structure data describes one or more protein structures, simulate interactions between a plurality of compounds and the one or more protein structures, determine binding affinities between each of the plurality of compounds and the one or more protein structures, and generate a list of validated or novel targets based on the binding affinities.

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom very large scale integrated (“VLSI”) circuits or gate arrays, off-the-shelf semiconductors such as logic chips, transistors, or other discrete components. A module may also be implemented in programmable hardware devices such as a field programmable gate array (“FPGA”), programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some embodiments, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods, and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C. As used herein, “a member selected from the group consisting of A, B, and C and combinations thereof” includes only A, only B, only C, a combination of A and B, a combination of B and C, a combination of A and C or a combination of A, B and C.

The present invention may be embodied in other specific forms without departing from its spirit or essential characteristics. The described embodiments are to be considered in all respects only as illustrative and not restrictive. The scope of the inventio is, therefore, indicated by the appended claims rather than by the foregoing description. All changes which come within the meaning and range of equivalency of the claims are to be embraced within their scope.

What is claimed is:

1. An apparatus, comprising:
at least one memory; and
at least one processor coupled with the at least one memory and configured to cause the apparatus to:
determine protein structure data associated with a plurality of genes, wherein the protein structure data describes one or more protein structures;
simulate interactions between a plurality of compounds and the one or more protein structures;
determine binding affinities between each of the plurality of compounds and the one or more protein structures; and
generate a list of validated or novel targets based on the binding affinities.

2. The apparatus of claim 1, wherein determining the protein structure data comprises retrieving protein sequence and structure files from a biological database.

3. The apparatus of claim 2, wherein the processor is configured cause the apparatus to select the structure files based on at least one of sequence coverage or resolution of available protein data.

4. The apparatus of claim 2, wherein determining the protein structure data comprises cleaning the structure files by removing water, heterogens, and side chains unrelated to the protein, and adding hydrogen atoms based on a predetermined pH value.

5. The apparatus of claim 1, wherein determining the protein structure data comprises selecting structure fragments for each of the plurality of genes using a coverage-resolution ranking algorithm.

6. The apparatus of claim 1, wherein simulating interactions comprises performing molecular docking using a docking engine configured with exhaustiveness and mode parameters.

7. The apparatus of claim 6, wherein simulating interactions comprises executing molecular docking computations in parallel across multiple processing units or servers.

8. The apparatus of claim 7, wherein the at least one processor is configured to cause the apparatus to aggregate docking scores from parallel docking simulations into a results file.

9. The apparatus of claim 1, wherein determining binding affinities comprises selecting, for each gene of the plurality of genes, a lowest docking score representing a most favorable binding configuration.

10. The apparatus of claim 9, wherein the at least one processor is configured to cause the apparatus to identify promiscuous targets based on a frequency of occurrence among top-ranked results for multiple compounds.

11. The apparatus of claim 10, wherein the at least one processor is configured to cause the apparatus to exclude promiscuous targets that appear among top-ranked targets for more than half of the plurality of compounds.

12. The apparatus of claim 1, wherein the at least one processor is configured to cause the apparatus to assign a statistical confidence score to each of the validated or novel targets based on variance among corresponding docking results.

13. The apparatus of claim 1, wherein the at least one processor is configured to cause the apparatus to apply a trained machine learning model configured to receive docking affinity data and output predicted off-target or toxic interactions based on the binding affinities.

14. The apparatus of claim 1, wherein the list of validated or novel targets comprises gene identifiers, compound identifiers, and binding affinity scores for each of the validated or novel targets.

15. The apparatus of claim 1, wherein the at least one processor is configured to cause the apparatus to store the list of validated or novel targets in a graph database linking compound identifiers to gene names and affinity metrics.

16. A method, comprising:
determining protein structure data associated with a plurality of genes, wherein the protein structure data describes one or more protein structures;
simulating interactions between a plurality of compounds and the one or more protein structures;
determining binding affinities between each of the plurality of compounds and the one or more protein structures; and
generating a list of validated or novel targets based on the binding affinities.

17. The method of claim 16, wherein determining the protein structure data comprises retrieving protein sequence and structure files from a biological database.

18. The method of claim 17, further comprising selecting the structure files based on at least one of sequence coverage or resolution of available protein data.

19. The method of claim 17, wherein determining the protein structure data comprises cleaning the structure files by removing water, heterogens, and side chains unrelated to the protein, and adding hydrogen atoms based on a predetermined pH value.

20. A computer program product comprising a non-transitory computer-readable medium storing instructions that, when executed by one or more processors, cause the one or more processors to:
determine protein structure data associated with a plurality of genes, wherein the protein structure data describes one or more protein structures;
simulate interactions between a plurality of compounds and the one or more protein structures;
determine binding affinities between each of the plurality of compounds and the one or more protein structures; and
generate a list of validated or novel targets based on the binding affinities.
